(19) United States
(12) Patent Application Publication
(10) Pub. No.: US 2021/0069045 A1
(43) Pub. Date: Mar. 11, 2021

(54) Differential-melting point PCM as safety indicator for warming devices

(71) Applicant: The Regents of the University of California, Oakland, CA (US)

(72) Inventors: Ashok Jagannath Gadgil, Berkeley, CA (US); Vi Hai Rapp, Berkeley, CA (US)

(73) Assignee: The Regents of the University of California, Oakland, CA (US)

(21) Appl. No.: 17/000,314

(22) Filed: Aug. 22, 2020

Related U.S. Application Data
(60) Provisional application No. 62/899,123, filed on Sep. 11, 2019.

(51) Int. Cl. A61G 11/00; A61F 7/02

(52) U.S. Cl. CPC A61G 11/00; A61F 2007/0095; A61F 2007/0292; A61F 7/02

(57) ABSTRACT

A robust, and inexpensive infant warmer comprises a predetermined temperature range indicator based on a phase change material (PCM) indicating suitable temperature for a given application

### INTRODUCTION

It is widely accepted that neonatal hypothermia remains an unsolved challenge that contributes to morbidity and mortality, especially in resource limited settings. The prevalence of neonatal hypothermia ranges widely depending on the definition of hypothermia and the patient population assessed, but was recently reported at 100% in a population of low birth weight infants in three low resource African settings. Those who are preterm, low birth weight, or ill are especially vulnerable to hypothermia.

Hypothermia can be prevented by providing a heat chain from the delivery room through inter/intra-hospital transport to the neonatal ward. In high-income countries, this heat chain is highly reliant on radiant warmers and incubators.

In resource limited settings where equipment, electricity and training can be inconsistent at best, these electrical sources of external heat may be unavailable or misused resulting in high rates of hypothermia, as well as concerns regarding hyperthermia and infection control. The equipment is expensive, requires a consistent source of electricity, and the lifespan can be short without adequate infrastructure to address complex maintenance and repairs. While provision of a heat chain remains necessary, it requires adaptation based on available resources.

The World Health Organization's (WHO) global standard is continuous Kangaroo Mother Care (KMC) to provide external heat. KMC is a well-established, effective practice with many benefits beyond heat provision, including improved lactation and bonding. However, there are times when KMC does not provide enough heat, when it is not feasible due to illness of the mother or infant, or when the mother needs to take a break to pursue activities that are not compatible with KMC, such as bathing, cooking, or attending to other critical economic or social needs. Other solutions currently available include hot water bottles, polyethylene wraps, bags, and mattresses filled with water, gels or Phase Change Materials (PCM). Each of these has major limitations related to safety, efficacy, and cost.

We developed a warmer designed specifically to address thermoregulation needs in the resource limited setting—a new technology for thermoregulation based on a phase-change mattress, specifically designed to complement KMC, to be intuitive to use with minimal training, and to be affordable and re-usable especially in settings without electricity thereby avoiding many of the limitations of currently available options. The Infant Warmer is a small mattress made of wax PCM that turns from liquid to solid at skin temperature (37° C.). The PCM is melted by being placed in a thermos of boiled water for approximately 30 minutes. It is then slipped into an insulating sleeve where it stays at goal temperature (37° C.) for approximately six hours. It is low cost, intuitive, re-usable, and non-electric. It can be used in the delivery room, neonatal ward, and on transport. The Infant Warmer can provide warmth when the patient is ill while still allowing easy accessibility for medical assessments and treatments. It is designed to be easily washable with standard hospital cleansers.

US20150066119 describes infant warmers with regulate temperature using a phase change material which changes between a liquid phase and a solid phase within a predetermined temperature range, and which comprise a temperature sensor such as a thermistor or thermocouple adjacent to the phase change material.

U.S. D773681 depicts a PCM-based Infant Warming Pad.

Nahimana et al., PHA 2018; 8(4): 211-217 disclose an Infant Warmer we tested in Rwanda using a liquid crystal thermometer (LCT) for temperature indication. The LCT devices failed after about 12 to 15 cycles of heating in boiling water and were also too expensive, making them unsuitable for eventual large-scale long-term use over many cycles of heating and cooling.

### SUMMARY OF THE INVENTION

Many times a warming device can be too warm for comfort, or even can cause injury. Thus, a warming device needs to provide reliable (often visual) indication that it is too hot for use. Our invention keeps an infant warmer safe, so that it does not scald a hypothermic infant that needs a warming pad. The invention provides methods, compositions and systems for a robust, reliable, detectable indication of a predetermined temperature range for a warming device.

In an embodiment the device comprises a small pouch enclosing a phase change material (PCM) selected so that it melts at a slightly higher temperature than intended use-temperature of the device. So when the indicator PCM is liquid, the device is too hot for use, and when it is solid, the device is not too hot to use.

In an aspect the invention provides a warming device comprising a predetermined temperature range indicator comprising a differential-melting point phase change material (PCM) wherein the indicator provides a visual and/or tactile signals for up and down deviations from the predetermined temperature range.

In embodiments:

the device is configured as an infant warmer;

the device is configured as a wearable infant warmer;

the device is configured as a wearable infant warmer, wherein the indicator is configured as transparent pouch that contains a color-dyed non-toxic indicator PCM that changes from liquid to solid at temperatures ranging from 40 C to 45 C;

the device is configured as a wearable infant warmer, comprising a mattress comprising sealed flexible compartments containing a wax PCM that turns from liquid to solid at skin temperature, wherein the indicator is integrated into the mattress;

the device is configured as a wearable infant warmer, comprising a mattress comprising sealed flexible compartments containing a wax PCM that turns from liquid to solid at skin temperature, wherein the indicator is integrated into the mattress, and is configured as transparent pouch that contains a color-dyed non-toxic indicator PCM that changes from liquid to solid at temperatures ranging from 40 C to 45 C;

the device is configured as a wearable infant warmer, comprising a heating mattress comprising sealed flexible compartments containing a clear PCM that solidifies at 38 C, wherein the indicator is integrated into the mattress, and is configured as transparent pouch that contains a color-dyed non-toxic indicator PCM that solidifies at 43 C, such that when the indicator-PCM solidifies, it signals to the user that the mat is safe for use, even when the clear PCM has not solidified;

the device is configured as a wearable infant warmer, comprising a mattress comprising a set of individual, parallel, elongate warming PCM candles, each in its own sleeve, wherein each sleeve is capped with an indicator enclosure, containing a color-dyed indicator PCM that changes from liquid to solid at temperatures ranging from 40 C to 45 C;

the device is configured as a wearable infant warmer, comprising a mattress comprising a set of individual, parallel, elongate warming PCM candles, each in its own sleeve, wherein each sleeve is capped with an indicator enclosure, containing a color-dyed indicator PCM that changes from liquid to solid at temperatures ranging from 40 C to 45 C, wherein the warming PCM candles turn from liquid to solid at skin temperature;

the indicator PCM solidifies at 43 C and the warming PCM solidifies at 38 C;

the mattress comprises twelve of the candles;

each candle comprises about 100 g of clear PCM;

the mattress measures about 45.7 cm, by 25.4 cm, by 1.91 cm thick and contains about 1.2 kilograms of warming PCM;

the sleeves are formed from co-extruded film made with polyethylene and nylon, stamped to form parallel enclosures for each candle;

each indicator enclosure contains about 10 g of red color-dyed PCM;

the sleeves are double-sleeved to ensure no leakage of PCM over many repeated cycles of heating and cooling;

the mattress is configured to be rolled up for heat charging or storage;

the mattress is immersed in heated water to charge (heat) the mattress and liquefy the warming PCM and the indicator PCM; and/or

the device is packaged in a sleeve with window or cutout configured to reveal one or more of the indicator enclosures.

In an aspect the invention provides methods of using the subject devices comprising warming an infant or item with the device by placing the device in thermal proximity to the infant or item.

The invention encompasses all combinations of the particular embodiments recited herein, as if each combination had been laboriously recited.

### BRIEF DESCRIPTION OF THE DRAWINGS

FIG. 1. Infant warming mat with liquid, red indicator-PCM that changes phase to solid at 43 C.

FIG. 2. Infant warming mat with solidified red indicator-PCM demonstrating that the mat is ready for use

FIG. 3. Infant warming mat with red indicator-PCM in nylon sleeve.

FIG. 4. Infant warming mat (with red indicator-PCM visible) wrapped in a blanket.

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. The examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

The invention provides robust, and inexpensive infant warmer that can perform reliably for thousands of cycles and comprising an indicator based on a Phase Change Material (PCM) indicating suitable temperature for a given application—not too hot and not too cold. Currently many such temperature indicators exist (e.g., those based on Liquid Crystal Temperature Indicators, electronic thermometers, etc.), however, none of them is inexpensive, extremely reliable over thousands of cycles, and affordably inexpensive for use in applications for resource-poor users (e.g., in low-income communities in the developing world).

In an embodiment of our novel approach, as applied to the Infant Warmer, the device to be monitored for temperature is attached to a transparent pouch or transparent enclosure that contains a color-dyed (e.g. red or orange) non-toxic indicator PCM that changes from liquid to solid at temperatures ranging from 40 C to 45 C. The indicator-PCM will signal (visual owing to transparency/opacity, or tactile owing to soft/hard) if the Infant Warmer is ready to use. The Infant Warmer is too hot to use when the indicator-PCM is liquid and transparent, and is safe to use when the indicator-PCM is solid and opaque. An example use of the indicator is shown below for an infant warming device.

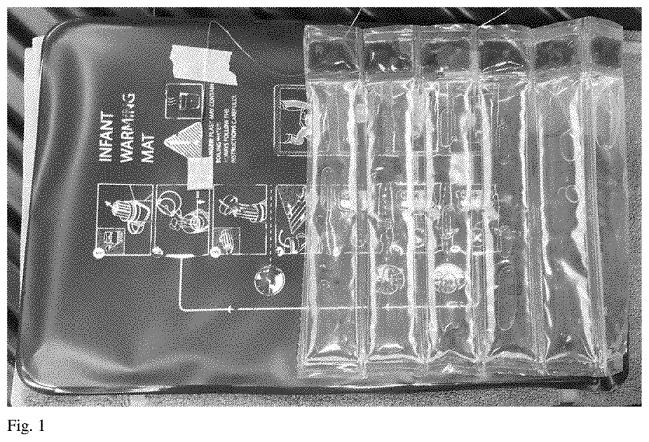

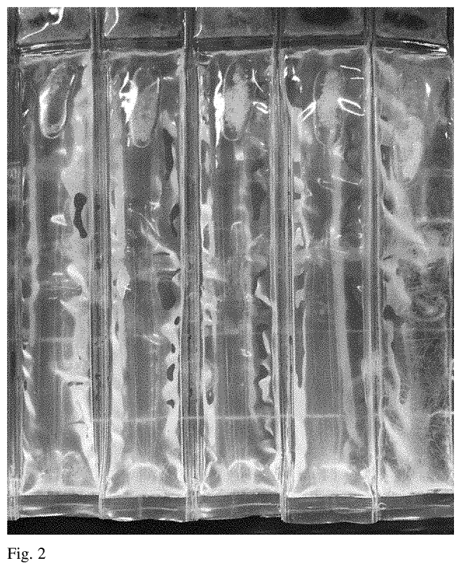

The infant warming device is heated with the indicator in boiling water until both the indicator and the warming material are liquid (see FIG. 1). The indicator-PCM (dyed red in the photographs) solidifies at 43 C. The main PCM the warming mat (white/clear in the photographs) contains a PCM that solidifies at 38 C, and is the main source of warmth for the hypothermic infant. In this example, when the indicator-PCM solidifies, it signals to the user that the mat is safe for use, even when the clear phase change material has not solidified (FIG. 2).

Advantages of our novel designs include: simple and easy to understand and use; non reliance on electricity; reliable, robust, and repeatable results over thousands of cycles; and relatively inexpensive compared to other ways to measure temperature reliably.

The invention may be embodied in a wide a variety of commercial applications, such as clothing, seating and bedding, mats, trays and pads (e.g. for stadium seating). Particular applications involve persons unable to effectively communicate that the device is too warm or too cold, such as infants, geriatrics, or persons with permanent or temporary disabilities. In an aspect the invention is incorporated in low-cost neonatal infant warmers. Because infants cannot complain or signal that the warmer is too hot to use, this is a critical part of the infant warmer. This can also be useful to all kinds of warmers for baby foods, warmer used for mothers' milk, and so on, where the recipient is unable to convey that the item is too hot. Other application include use in geriatric and convalescent facilities, as infant warming mats to prevent hypothermia, warming mats for alleviating pain (e.g. from injuries, menstruation, etc.). The invention may be integrated in other types of body warmers, such as heating pads, heating socks, glove, hats, boots, etc. In a particular embodiment the invention is integrated in a warming device configured for a patient in need of increased peripheral circulation, particularly in an extremity such as a foot.

In the specific application for an Infant Warmer, the PCM-based “Hot indicator” provides easy to understand visual and tactile signal if the Infant Warmer exceeds the maximum temperature beyond which it can cause harm to the infant. This indicator design consistently indicates warming temperature and was invented to replace indicators that may be used for warming devices in resource-constrained conditions. The indicator-PCM is initiated by melting the phase change material using boiling water or other means (e.g. an oven) well exceeding the desired use temperature, e.g., 100 C. After the indicator-PCM and warmer-PCM have both fully melted, the Infant Warmer is removed from the warming environment. The user will immediately determine if the warming device is ready by inspecting the indicator. The Infant Warmer will be ready for use with the indicator-PCM has frozen solid, but the main PCM is still liquid. If the indicator-PCM is still fully or partially liquid, then the warming device is still too hot for use.

Suitable PCMs include paraffin oils and naturally occurring fatty acids and esters, including vegetable oils, plant oils, algal oils, animal fats, tall oil fatty acid, or a combination of any two or more thereof. Source materials include chicken fat, beef tallow, yellow grease, choice white grease, lard, soybean oil, corn oil, cottonseed oil, canola oil, coconut oil, sunflower oil, palm oil, palm kernel oil, rapeseed oil, or a combination of any two or more thereof. In embodiments the PCMs comprise one or more hydrogenated plant oil, such as palm oil, palm kernel oil, rapeseed oil, coconut oil and soybean oil, including proprietary formulations such as PureTemp37 for the warming PCM and PureTemp44 for the indicator PCM (PureTemp LLC, MN)

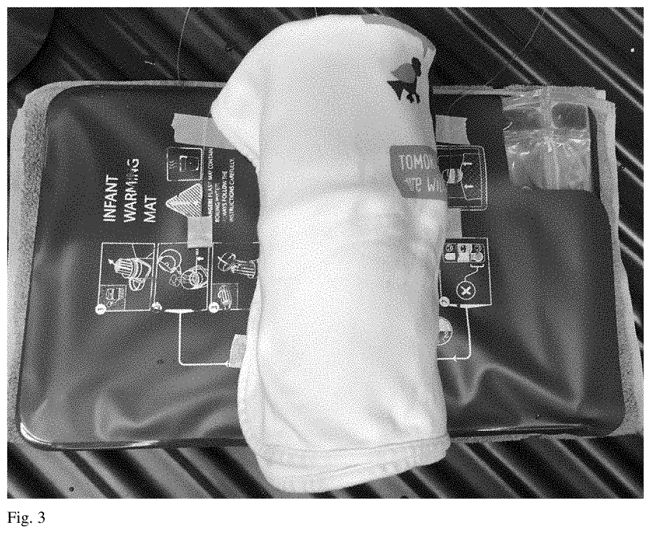

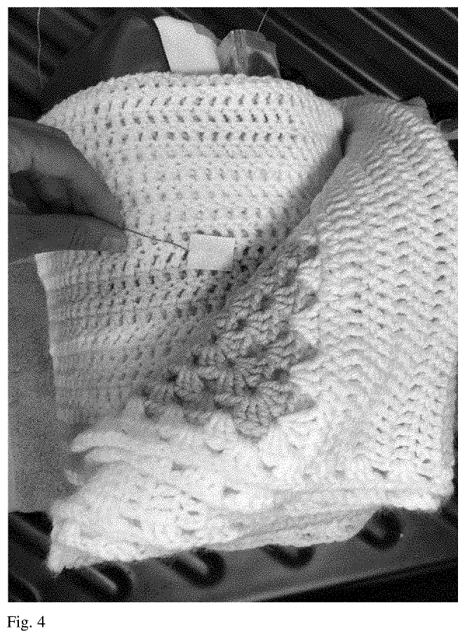

In an embodiment (see, FIGS. 1-4) the heating component of the infant warmer is a mattress comprised of a set of individual phase-change material (PCM) candles, each in its own sleeve. In an embodiment, the mattress comprises 12 candles made of PureTemp 37 wax (Entropy Solutions, Minneapolis, Minn., USA). Each individual candle holds about 100 g of PCM. This mat measures about 45.7 cm, by 25.4 cm, by 1.91 cm thick and contains about 1.2 kilograms of PCM. The casing for the PCM is a co-extruded film made with polyethylene and nylon, stamped to form parallel enclosures for each candle. Each candle enclosure is capped with a corresponding indicator enclosure. In an embodiment each indicator enclosure contains about 10 g of red color-dyed PCM of PureTemp44, providing visual and tactile signals for up and down deviations from the predetermined temperature range. The construction may be double-sleeved to ensure no leakage of PCM over many repeated cycles of heating and cooling. Preferred configurations allow the mat to be rolled up for charging or storage. Boiling water is used to charge the mat. Additional details for the base mattress design are disclosed in U.S. Pat. No. D773681.

1. A warming device comprising a predetermined temperature range indicator comprising a differential-melting point phase change material (PCM) wherein the indicator provides visual and tactile signals for up and down deviations from the predetermined temperature range.

2. The warming device of claim 1, configured as an infant warmer.

3. The warming device of claim 1, configured as a wearable infant warmer.

4. The warming device of claim 1, configured as a wearable infant warmer, wherein the indicator is configured as transparent pouch that contains a color-dyed non-toxic indicator PCM that changes from liquid to solid at temperatures ranging from 40 C to 45 C.

5. The warming device of claim 1, configured as a wearable infant warmer, comprising a mattress comprising sealed flexible compartments containing a wax PCM that turns from liquid to solid at skin temperature, wherein the indicator is integrated into the mattress.

6. The warming device of claim 1, configured as a wearable infant warmer, comprising a mattress comprising sealed flexible compartments containing a wax PCM that turns from liquid to solid at skin temperature, wherein the indicator is integrated into the mattress, and is configured as transparent pouch that contains a color-dyed non-toxic indicator PCM that changes from liquid to solid at temperatures ranging from 40 C to 45 C.

7. The warming device of claim 1, configured as a wearable infant warmer, comprising a heating mattress comprising sealed flexible compartments containing a clear PCM that solidifies at 38 C, wherein the indicator is integrated into the mattress, and is configured as transparent pouch that contains a color-dyed non-toxic indicator PCM that solidifies at 43 C, such that when the indicator-PCM solidifies, it signals to the user that the mat is safe for use, even when the clear PCM has not solidified.

8. The warming device of claim 1, configured as a wearable infant warmer, comprising a mattress comprising a set of individual, parallel, elongate warming PCM candles, each in its own sleeve, wherein each sleeve is capped with an indicator enclosure, containing a color-dyed indicator PCM that changes from liquid to solid at temperatures ranging from 40 C to 45 C.

9. The warming device of claim 1, configured as a wearable infant warmer, comprising a mattress comprising a set of individual, parallel, elongate warming PCM candles, each in its own sleeve, wherein each sleeve is capped with an indicator enclosure, containing a color-dyed indicator PCM that changes from liquid to solid at temperatures ranging from 40 C to 45 C, wherein the warming PCM candles turn from liquid to solid at skin temperature.

10. The warming device of claim 9 wherein indicator PCM solidifies at 43 C and the warming PCM solidifies at 38 C.

11. The warming device of claim 9 wherein the mattress comprises twelve of the candles.

12. The warming device of claim 9 wherein each candle comprises about 100 g of clear PCM.

13. The warming device of claim 9 wherein the mattress measures about 45.7 cm, by 25.4 cm, by 1.91 cm thick and contains about 1.2 kilograms of warming PCM.

14. The warming device of claim 9 wherein the sleeves are formed from co-extruded film made with polyethylene and nylon, stamped to form parallel enclosures for each candle.

15. The warming device of claim 9 wherein each indicator enclosure contains about 10 g of red color-dyed PCM.

16. The warming device of claim 9 wherein the sleeves are double-sleeved to ensure no leakage of PCM over many repeated cycles of heating and cooling.

17. The warming device of claim 9 wherein the mattress is configured to be rolled up for heat charging or storage.

18. The warming device of claim 9 wherein the mattress is immersed in heated water to charge (heat) the mattress and liquefy the warming PCM and the indicator PCM.

19. The warming device of claim 9, packaged in a sleeve with window or cutout configured to reveal one or more of the indicator enclosures.

20. A method of using a disclosed warming device comprising warming an infant or item with the device by placing the device in thermal proximity to the infant or item.
